(19) United States
(12) Patent Application Publication
(10) Pub. No.: US 2012/0122920 A1
(43) Pub. Date: May 17, 2012

(54) LIBRARIES OF N-SUBSTITUTED-N-PHENYLETHYLSULFONAMIDES FOR DRUG DISCOVERY

(76) Inventors: JOSEP CASTELLS BOLIART, Mollet Del Valles (ES); David Enrique MIGUEL CENTENO, Mollet Del Valles (ES); Marta PASCUAL GILABERT, Mollet Del Valles (ES)

(21) Appl. No.: 13/334,379

(22) Filed: Dec. 22, 2011

Related U.S. Application Data
(63) Continuation-in-part of application No. PCT/ES2010/070434, filed on Jun. 28, 2010.

(30) Foreign Application Priority Data
Jun. 26, 2009 (ES) P200901525

(51) Int. Cl. A61K 31/47; A61K 31/18; A61K 31/445; A61P 7/00; A61K 31/381; A61K 31/404; C40B 40/04; A61P 29/00

(52) U.S. Cl. 514/311; 506/15; 514/604; 514/602; 514/603; 514/445; 514/415; 514/331

(57) ABSTRACT

New compounds are continually being sought for the treatment and prevention of disorders. The invention relates to N-substituted-N-phenylethylsulfonamides libraries which can be used in the search for, and identification of, new lead compounds that could modulate the functional activity of a biological target.

This application is a continuation-in-part of and claims priority under 35 U.S.C. 365(c) from PCT/ES2010/070434, filed 28 Jun. 2010, the disclosure of which is incorporated by reference herein.

### FIELD OF THE INVENTION

The field of the invention is medicinal chemistry. The invention relates to N-substituted-N-phenylethylsulfonamides which can be therapeutically active.

### BACKGROUND OF THE INVENTION

Novel compounds are continually sought after to treat and prevent diseases and disorders. Pharmaceutical companies interested in developing new active molecules develop and purchase libraries of chemical compounds in order to screen their biological or pharmacological activity against a particular target, aiming at the identification of new industrially useful products. Therefore, there is a market of customer companies for which the acquisition of novel libraries of chemical compounds, not already biologically explored, is a key issue. Therefore, for the companies whose core business is the design and preparation of libraries of chemical compounds, their commercialization has a clear industrial interest.

In the context of the present invention, “library” is applied to a group of compounds which are structurally related by a main base structure (scaffold), but which are distinguishable by the changes in the specific substitute groups linked to the base structure.

Although many research groups work to find novel compounds to be used in the treatment of known or novel diseases, the number of active new chemical entities in the market doesn't grow in the same extension. Over the past few years, there has been a progressive reduction in the number of medicines entering the market mainly due to the more stringent regulatory requirements that have raised the bar on safety and efficacy of new drugs.

The libraries of compounds described in this invention are useful for contributing to the search and identification of new lead compounds which can modulate the functional activity of a biological target. In addition, the libraries allow to explore the chemical space, to increase the structural diversity of valuable molecules in the pharmaceutical sector and to increase the elements of structural recognition in order to study their interaction with biological targets of interest in the pharmaceutical or medical chemistry field. For instance, the molecules may be therapeutically useful as anti-inflammatory or anticoagulation agents, among many other applications.

The present invention is useful for a systematic synthesis of large libraries of compounds with industrial applicability. The present invention is useful for making libraries and subsequently optimising the compounds considered as relevant according to the target of interest.

Libraries described in this invention are useful for being biologically and pharmaceutically explored, and therefore to contribute in the research and identification of new drug leads able to modulate the functional activity of a biological target, since these molecules are new sources of chemical diversity not explored up to date. The libraries of the present invention can be explored by means of any known method of biological screening. These methods comprise, but are not limited to, receptor affinity assays, ELISA assays, “southern”, “western” and “northern blot”, and competitive binding assays.

U.S. Pat. No. 7,126,006 B2 (The Scripps Research Institute) describes glycoluryl type molecules as scaffolds in the preparation of combinatorial libraries.

U.S. Pat. No. 6,939,973 B1 (The Scripps Research Institute) describes glycoluryl type molecules as scaffolds in the preparation of combinatorial libraries.

The search for novel drug lead compounds for drug discovery is a difficult task that has traditionally required the use of hundreds of thousands of compounds to reach a successful molecule, mainly due to the fact that drug discovery was driven by random screening and the chemical and biological intuition.

However, integrated approaches combining structural knowledge from conformationally constrained small peptides and parallel synthesis of small molecules are particulary well suited for the shortening of the time-consuming drug discovery process.

Compounds of formula (I) have been designed using computational techniques such as virtual library screening based on pharmacophore search. Virtual (database) screening (VS) is an important component of the computer-based search of novel lead compounds. The primary VS premise is to screen a database of molecules computationally using structural descriptors that relate in some way to potential biological activity. A subset of database molecules found to match these descriptors can then be selected for subsequent biological analysis. In terms of novel lead discovery, pharmacophore searching is one of the most widely applied VS methods.

Compounds of formula (I) are not an arbitrary selection of a vast amount of molecules. On the contrary, they have been designed using as starting point a pharmacophore for at least BK antagonism. In this context, a pharmacophore is defined as a critical arrangement of molecular fragments or features creating a necessary, although not sufficient, condition for biological activity and receptor affinity.

In order to improve the success of molecular bioactive conformations, applicants have defined the structure of compounds of formula (I) using a pharmacophore based on Hoe 140, the most potent peptide antagonist of bradykinin (BK, sequence: D-Arg⁰-Arg¹-Pro²-Hyp³-Gly⁴-Thi⁵-Ser⁶-D-Tic⁷-Oic⁸-Arg⁹ (Hyp, hydroxyproline; Thi, β-(2-thienyl)-alanine; Tic, 1,2,3,4-tetrahydroisoquinoline-3-carboxylic acid; Oic, (2S,3aS,7aS)-octahydroindole-2-carboxylic acid). The pharmacophore for BK antagonism has been obtained from a conformational search using an iterative simulated annealing procedure. Corcho, F J. Computational Studies on the Structure and Dynamics of Bioactive Peptides, PhD Thesis, 2004.

In conclusion, all compounds of formula (I) exhibit at least Hoe 140 pharmacophore fulfillment, and therefore they share specific characteristics for receptor affinity critical in the search of novel bioactive molecules.

### DESCRIPTION OF THE INVENTION

The present invention concerns libraries of chemical compounds wherein each member of said library is a compound of formula (I)

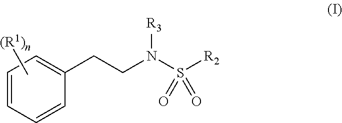

and the salts and stereoisomers thereof, wherein
R¹ is hydrogen, halo, hydroxy, nitro, cyano, carboxyl, C₁₋₆alkyl, C₁₋₆alkoxy, C₁₋₆alkoxyC₁₋₆alkyl, C₁₋₆alkylcarbonyl, amino, mono- or diC₁₋₆alkylamino, azido, mercapto, polyhaloC₁₋₆alkyl, and polyhaloC₁₋₆alkoxy, aryl, Het;
R² is C₁₋₆alkyl, C₁₋₄ alkylcarbonyl, C₁₋₆alkyl optionally substituted with aryl, C₁₋₆alkoxyC₁₋₆alkyl, or C₃₋₇cycloalkyl, C₁₋₆alkyl optionally substituted with Het, C₃₋₇cycloalkyl optionally substituted with C₁₋₆alkyl, C₁₋₆alkyl optionally substituted with C₃₋₇cycloalkyl, aryl or Het, C₂₋₆alkenyl optionally substituted with C₃₋₇cycloalkyl or aryl; aryl; Het;
R³ is C₁₋₆alkyl, C₁₋₆alkoxy, C₁₋₆alkoxyC₁₋₆alkyl, C₁₋₆alkylcarbonyl, mono- or diC₁₋₆alkylamino, polyhaloC₁₋₆alkyl, and polyhaloC₁₋₆alkoxy, C₁₋₄ alkoxyC₁₋₆alkyl, or C₃₋₇cycloalkyl, C₁₋₆alkyl optionally substituted with C₃₋₇cycloalkyl or aryl, C₁₋₆alkyl optionally substituted with Het, C₃₋₇cycloalkyl optionally substituted with C₁₋₆alkyl, C₂₋₆alkenyl optionally substituted with C₃₋₇cycloalkyl, aryl or Het; aryl, Het; C₁₋₆alkyl optionally substituted with —NR^{4a}R^{4b}, wherein R^{4a} and R^{4b} are, each independently, C₁₋₆alkyl, or R^{4a} and R^{4b} together with the nitrogen to which they are attached form a 5- or 6-membered saturated heterocyclic ring;
n is one, two, three, four or five;
each aryl as a group or part of a group is phenyl or naphthalenyl, each optionally substituted with one, two or three substituents selected from halo, hydroxy, nitro, cyano, carboxyl, C₁₋₆alkyl, C₁₋₆alkoxy, C₁₋₆alkoxyC₁₁ alkyl, C₁₋₆alkylcarbonyl, amino, mono- or diC₁₋₆alkylamino, azido, mercapto, polyhaloC₁₋₆alkyl, and polyhaloC₁₋₆alkoxy; and
each Het as a group or part of a group is a monocyclic ring with 5 or 6 ring atoms or a bicyclic ring structure comprising a 6 membered ring fused to a 4, 5, or 6 membered ring; each of the rings being saturated, partially unsaturated, or completely unsaturated; at least one of the rings containing 1 to 4 heteroatoms each independently selected from nitrogen, oxygen and sulphur; and any one of the rings being optionally substituted with one, two or three substituents each independently selected from the group consisting of halo, hydroxy, nitro, cyano, carboxyl, C₁₋₆alkyl, C₁₋₆alkoxy, C₁₋₆alkoxyC₁₋₆alkyl, C₁₋₆alkylcarbonyl, amino, mono- or diC₁₋₆alkylamino, azido, mercapto, polyhaloC₁₋₆alkyl, polyhaloC₁₋₆alkoxy, and C₃₋₇cycloalkyl.

The invention further relates to methods for the preparation of the libraries of compounds where each member of said library is a compound of formula (I), the N-oxides, addition salts, quaternary amines, metal complexes, and stereochemically isomeric forms thereof, their intermediates, and the use of the intermediates in the preparation of the compounds of formula (I).

The invention relates to the libraries of compounds of formula (I) per se, the N-oxides, addition salts, quaternary amines, metal complexes, and stereochemically isomeric forms thereof, for use as a drug. In addition, the present invention relates to pharmaceutical compositions including the compounds mentioned above for being administrated to patients for the treatment of inflammation.

The invention also relates to the use of libraries of compounds of formula (I), or N-oxide, addition salt, quaternary amine, metallic complex and stereochemically isomeric forms thereof, for the manufacture of a medicament for the treatment of a disease or conditions such as inflammation or coagulation. Likewise, the present invention relates to the use of the compound of formula (I), or N-oxide, addition salt, quaternary amine, metallic complex and stereochemically isomeric forms thereof, for use in the treatment of a disease or conditions such as inflammation or coagulation. Or the invention relates to a method for treating a disease or conditions such as inflammation or coagulation in a warm-blooded animal, said method comprising administering an effective amount of compound of formula (I), or N-oxide, addition salt, quaternary amine, metallic complex and stereochemically isomeric forms thereof.

From now onwards in the present specification, when using the term “libraries of compounds of formula (I) or “libraries of compounds where each member of the library is a compound of formula (I)” or “the present libraries” or “the present compounds” or similar terms, is meant to include in the libraries of compounds of formula (I), all and each of the subgroups thereof, N-oxides, addition salts, quaternary amines, metallic complex and stereochemically isomeric forms.

The present disclosure also includes the prodrugs of compounds of formula (I).

As used in the foregoing and hereinafter, the following definitions apply unless otherwise noted.

The term halo is generic to fluoro, chloro, bromo and iodo.

The term “polyhaloC₁₋₆alkyl” as a group or part of a group, e.g. in polyhaloC₁₋₆alkoxy, is defined as mono- or polyhalo substituted C₁₋₆alkyl, in particular C₁₋₆alkyl substituted with up to one, two, three, four, five, six, or more halo atoms, such as methyl or ethyl with one or more fluoro atoms, for example, difluoromethyl, trifluoromethyl, trifluoroethyl. Preferred is trifluoromethyl. Also included are perfluoroC₁₋₆alkyl groups, which are C₁₋₆alkyl groups wherein all hydrogen atoms are replaced by fluorine atoms, e.g. pentafluoroethyl. In case more than one halogen atom is attached to an alkyl group within the definition of polyhaloC₁₋₆alkyl, the halogen atoms may be the same or different.

As used herein “C₁₋₄alkyl” as a group or part of a group defines straight or branched chain saturated hydrocarbon radicals having from 1 to 4 carbon atoms such as for example methyl, ethyl, 1-propyl, 2-propyl, 1-butyl, 2-butyl, 2-methyl-1-propyl; “C₁₋₆alkyl” encompasses C₁₋₄alkyl radicals and the higher homologues thereof having 5 or 6 carbon atoms such as, for example, 1-pentyl, 2-pentyl, 3-pentyl, 1-hexyl, 2-hexyl, 2-methyl-1-butyl, 2-methyl-1-pentyl, 2-ethyl-1-butyl, 3-methyl-2-pentyl, and the like. Of interest amongst C₁₋₆alkyl is C₁₋₄alkyl.

The term “C₂₋₆alkenyl” as a group or part of a group defines straight and branched chained hydrocarbon radicals having saturated carbon-carbon bonds and one double bond, and having from 2 to 6 carbon atoms, such as, for example, ethenyl (or vinyl), 1-propenyl, 2-propenyl (or allyl), 1-butenyl, 2-butenyl, 3-butenyl, 2-methyl-2-propenyl, 2-pentenyl, 3-pentenyl, 2-hexenyl, 3-hexenyl, 4-hexenyl, 2-methyl-2-butenyl, 2-methyl-2-pentenyl and the like. Of interest amongst C₂₋₆alkenyl is C₂₋₄ alkenyl.

C₃₋₇cycloalkyl is generic to cyclopropyl, cyclobutyl, cyclopentyl, cyclohexyl and cycloheptyl.

C₁₋₆alkoxy means C₁₋₆alkyloxy wherein C₁₋₆alkyl is as defined above.

It should be noted that the radical positions on any molecular moiety used in the definitions may be anywhere on such moiety as long as it is chemically stable.

Radicals used in the definitions of the variables include all possible positional isomers unless otherwise indicated. For instance pyridyl includes 2-pyridyl, 3-pyridyl and 4-pyridyl; pentyl includes 1-pentyl, 2-pentyl and 3-pentyl.

When any variable occurs more than one time in any constituent, each definition is independent.

An embodiment of the present invention relates to libraries of compounds of formula (I) or any subgroup of compounds of formula (I) of the invention, as well as N-oxides, salts, and possible stereochemical forms thereof. Another embodiment relates to libraries of compounds of formula (I) o any subgroup of compounds of formula (I) disclosed herein, as well as salts, and possible stereochemical forms thereof.

The compounds of formula (I) making up the libraries of the present invention may have one or more centers of chirality and may exist as stereochemically isomeric forms. The term “stereochemically isomeric forms” as used herein defines all the possible compounds made up of the same atoms bonded by the same sequence of atoms but having different three-dimensional structures which are not interchangeable, which the compounds of formula (I) may possess.

With reference to the instances where (R) or (S) is used to designate the absolute configuration of a chiral atom within a substituent, the designation is done taking into consideration the whole compound and not the substituent in isolation.

Unless otherwise mentioned or indicated, the chemical designation of a compound encompasses the mixture of all possible stereochemically isomeric forms, which said compound may possess. Said mixture may contain all diastereomers and/or enantiomers of the basic molecular structure of said compound. All stereochemically isomeric forms of the compounds of the present invention both in pure form or mixed with each other are intended to be embraced within the scope of the present invention.

Pure stereoisomeric forms of the compounds and intermediates as mentioned herein are defined as isomers substantially free of other enantiomeric or diastereomeric forms of the same basic molecular structure of said compounds or intermediates. In particular, the term “stereoisomerically pure” concerns compounds or intermediates having a stereoisomeric excess of at least 80% (i.e. minimum 90% of one isomer and maximum 10% of the other possible isomers) up to a stereoisomeric excess of 100% (i.e. 100% of one isomer and none of the other), more in particular, compounds or intermediates having a stereoisomeric excess of 90% up to 100%, even more in particular having a stereoisomeric excess of 94% up to 100% and most in particular having a stereoisomeric excess of 97% up to 100%. The terms “enantiomerically pure” and “diastereomerically pure” should be understood in a similar way, but then having regard to the enantiomeric excess, and the diastereomeric excess, respectively, of the mixture in question.

Pure stereoisomeric forms of the compounds and intermediates of the libraries of this invention may be obtained by the application of art-known procedures. For instance, enantiomers may be separated from each other by the selective crystallization of their diastereomeric salts with optically active acids or bases. Examples thereof are tartaric acid, dibenzoyltartaric acid, ditoluoyltartaric acid and camphorsulfonic acid. Alternatively, enantiomers may be separated by chromatographic techniques using chiral stationary phases. Said pure stereochemically isomeric forms may also be derived from the corresponding pure stereochemically isomeric forms of the appropriate starting materials, provided that the reaction occurs stereospecifically. Preferably, if a specific stereoisomer is desired, said compound will be synthesized by stereospecific methods of preparation. These methods will advantageously employ enantiomerically pure starting materials.

The diastereomeric racemates of the compounds of formula (I) can be obtained separately by conventional methods. Appropriate physical separation methods that may advantageously be employed are, for example, selective crystallization and chromatography, e.g. column chromatography.

For some of the compounds of formula (I), their N-oxides, salts, solvates, quaternary amines, or metal complexes, and the intermediates used in the preparation thereof, the absolute stereochemical configuration was not experimentally determined. A person skilled in the art is able to determine the absolute configuration of such compounds using art-known methods such as, for example, X-ray diffraction.

The present invention is also intended to include all isotopes of atoms occurring on the present compounds. Isotopes include those atoms having the same atomic number but different mass numbers. By way of general example and without limitation, isotopes of hydrogen include tritium and deuterium. Isotopes of carbon include C-13 and C-14.

The term “prodrug” as used throughout this text means the pharmacologically acceptable derivatives such as esters, amides, and phosphates, such that the resulting in vivo biotransformation product of the derivative is the active drug as defined in the compounds of formula (I). The reference by Goodman and Gilman (The Pharmacological Basis of Therapeutics, 8th ed, McGraw-Hill, Int. Ed. 1992, “Biotransformation of Drugs”, p 13-15) describing prodrugs generally is hereby incorporated. Prodrugs preferably have excellent aqueous solubility, increased bioavailability and are readily metabolized into the active inhibitors in vivo. Prodrugs of a compound of the present invention may be prepared by modifying functional groups present in the compound in such a way that the modifications are cleaved, either by routine manipulation or in vivo, to the parent compound.

Preferred are pharmaceutically acceptable ester prodrugs that are hydrolysable in vivo and are derived from those compounds of formula (I) having a hydroxy or a carboxyl group. An in vivo hydrolysable ester is an ester, which is hydrolysed in the human or animal body to produce the parent acid or alcohol. Suitable pharmaceutically acceptable esters for carboxy include C₁₋₆alkoxymethyl esters for example methoxymethyl, C₁₋₆alkanoyloxymethyl esters for example pivaloyloxymethyl, phthalidyl esters, C₃₋₈cycloalkoxycarbonyloxyC₁₋₆alkyl esters for example 1-cyclohexylcarbonyloxyethyl; 1,3-dioxolen-2-onylmethyl esters for example 5-methyl-1,3-dioxolen-2-onylmethyl; and C₁₋₆alkoxycarbonyloxyethyl esters for example 1-methoxycarbonyl-oxyethyl which may be formed at any carboxy group in the compounds of this invention.

An in vivo hydrolysable ester of a compound of the formula (I) containing a hydroxy group includes inorganic esters such as phosphate esters and α-acyloxyalkyl ethers and related compounds which as a result of the in vivo hydrolysis of the ester breakdown to give the parent hydroxy group. Examples of α-acyloxyalkyl ethers include acetoxymethoxy and 2,2-dimethylpropionyloxy-methoxy. A selection of in vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl. Examples of substituents on benzoyl include morpholino and piperazino linked from a ring nitrogen atom via a methylene group to the 3- or 4-position of the benzoyl ring.

For therapeutic use, salts of the compounds of formula (I) are those wherein the counter-ion is pharmaceutically acceptable. However, salts of acids and bases which are non-pharmaceutically acceptable may also find use, for example, in the preparation or purification of a pharmaceutically acceptable compound. All salts, whether pharmaceutically acceptable or not are included within the ambit of the present invention.

The pharmaceutically acceptable acid and base addition salts as mentioned hereinabove are meant to comprise the therapeutically active non-toxic acid and base addition salt forms which the compounds of formula (I) are able to form. The pharmaceutically acceptable acid addition salts can conveniently be obtained by treating the base form with such appropriate acid. Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic (i.e. ethanedioic), malonic, succinic (i.e. butanedioic acid), maleic, fumaric, malic (i.e. hydroxybutanedioic acid), tartaric, citric, methanesulfonic, ethanesulfonic, benzenesulfonic, p-toluenesulfonic, cyclamic, salicylic, p-aminosalicylic, pamoic and the like acids.

Conversely said salt forms can be converted by treatment with an appropriate base into the free base form.

The compounds of formula (I) containing an acidic proton may also be converted into their non-toxic metal or amine addition salt forms by treatment with appropriate organic and inorganic bases. Appropriate base salt forms comprise, for example, the ammonium salts, the alkali and earth alkaline metal salts, e.g. the lithium, sodium, potassium, magnesium, calcium salts and the like, salts with organic bases, e.g. the benzathine, N-methyl-D-glucamine, hydrabamine salts, and salts with amino acids such as, for example, arginine, lysine and the like.

The term addition salt as used hereinabove also comprises the solvates which the compounds of formula (I) as well as the salts thereof, are able to form. Such solvates are for example hydrates, alcoholates and the like.

The term “quaternary amine” as used hereinbefore defines the quaternary ammonium salts which the compounds of formula (I) are able to form by reaction between a basic nitrogen of a compound of formula (I) and an appropriate quaternizing agent, such as, for example, an optionally substituted alkylhalide, arylhalide or arylalkylhalide, e.g. methyliodide or benzyliodide. Other reactants with good leaving groups may also be used, such as alkyl trifluoromethanesulfonates, alkyl methanesulfonates, and alkyl p-toluenesulfonates. A quaternary amine has a positively charged nitrogen. Pharmaceutically acceptable counterions include chloro, bromo, iodo, trifluoroacetate and acetate. The counterion of choice can be introduced using ion exchange resins.

The N-oxide forms of the present compounds are meant to comprise the compounds of formula (I) wherein one or several nitrogen atoms are oxidized to the so-called N-oxide.

It will be appreciated that the compounds of formula (I) may have metal binding, chelating, complex forming properties and therefore may exist as metal complexes or metal chelates. Such metalated derivatives of the compounds of formula (I) are intended to be included within the scope of the present invention.

Some of the compounds of formula (I) may also exist in their tautomeric form. Such forms although not explicitly indicated in the above formula are intended to be included within the scope of the present invention.

One embodiment of the present invention relates to libraries of compounds of formula (I) or of any subgroup of compounds of formula (I), wherein one or more of the following conditions apply:
R¹ is hydrogen, aryl, Het;
R² is C₁₋₆alkyl optionally substituted with aryl, C₁₋₆alkyl optionally substituted with Het, C₃₋₇cycloalkyl optionally substituted with C₁₋₆alkyl, C₁₋₆alkyl optionally substituted with C₃₋₇cycloalkyl or aryl, C₂₋₆alkenyl optionally substituted with C₃₋₇cycloalkyl, aryl or Het; aryl; Het;
R³ is C₁₋₆alkyl, C₁₋₆alkoxy, C₁₋₆alkoxyC₁₋₆alkyl, C₁₋₆alkylcarbonyl, polyhaloC₁₋₆alkyl, and polyhaloC₁₋₆alkoxy, C₁₋₆alkoxyC₁₋₆alkyl, C₃₋₇cycloalkyl, C₁₋₆alkyl optionally substituted with C₃₋₇cycloalkyl or aryl, C₁₋₆alkyl optionally substituted with Het, C₃₋₇cycloalkyl optionally substituted with C₁₋₆alkyl, C₂₋₆alkenyl optionally substituted with C₃₋₇cycloalkyl, aryl or Het; aryl; Het;
n is one or two;
each aryl as a group or part of a group is phenyl or naphthalenyl, each optionally substituted with one, two or three substituents selected from halo, hydroxy, nitro, cyano, carboxyl, C₁₋₆alkyl, C₁₋₆alkoxy, C₁₋₆alkoxyC₁₋₆alkyl, C₁₋₆alkylcarbonyl, amino, mono- or diC₁₋₆alkylamino, azido, mercapto, polyhaloC₁₋₆alkyl, and polyhaloC₁₋₆alkoxy; and
each Het as a group or part of a group is a monocyclic ring with 5 or 6 ring atoms or a bicyclic ring structure comprising a 6 membered ring fused to a 4, 5, or 6 membered ring; each of the rings being saturated, partially unsaturated, or completely unsaturated; at least one of the rings containing 1 to 4 heteroatoms each independently selected from nitrogen, oxygen and sulphur; and any one of the rings being optionally substituted with one, two or three substituents each independently selected from the group consisting of halo, hydroxy, nitro, cyano, carboxyl, C₁₋₆alkyl, C₁₋₆alkoxy, C₁₋₆alkoxyC₁₋₆alkyl, C₁₋₆alkylcarbonyl, amino, mono- or diC₁₋₆alkylamino, azido, mercapto, polyhaloC₁₋₆alkyl, polyhaloC₁₋₆alkoxy, and C₃₋₇cycloalkyl.

One embodiment of the present invention relates to libraries of compounds of formula (I) or of any subgroup of compounds of formula (I), wherein one or more of the following conditions apply:
R¹ is hydrogen;
R² is aryl or Het;
R³ is C₁₋₆alkyl, C₁₋₆alkoxy, C₁₋₆alkoxyC₁₋₆alkyl, C₁₋₆alkylcarbonyl, mono- or diC₁₋₆alkylamino, polyhaloC₁₋₆alkyl, and polyhaloC₁₋₆alkoxy, C₁₋₆alkoxyC₁₋₆alkyl, or C₃₋₇cycloalkyl, C₁₋₆alkyl optionally substituted with C₃₋₇cycloalkyl or aryl, C₁₋₆alkyl optionally substituted with Het, C₃₋₇cycloalkyl optionally substituted with C₁₋₆alkyl, C₂₋₆alkenyl optionally substituted with C₃₋₇cycloalkyl, aryl or Het; aryl, Het, C₁₋₆alkyl optionally substituted with —NR^{4a}R^{4b}, wherein R^{4a} and R^{4b} are, each independently, C₁₋₆alkyl, or R^{4a} and R^{4b} together with the nitrogen to which they are attached form a 5- or 6-membered saturated heterocyclic ring;
n is one;
each aryl as a group or part of a group is phenyl or naphthalenyl, each optionally substituted with one, two or three substituents selected from halo, hydroxy, nitro, cyano, carboxyl, C₁₋₆alkyl, C₁₋₆alkoxy, C₁₋₆alkoxyC₁₋₆alkyl, C₁₋₆alkylcarbonyl, amino, mono- or diC₁₋₆alkylamino, azido, mercapto, polyhaloC₁₋₆alkyl, and polyhaloC₁₋₆alkoxy; and
each Het as a group or part of a group is a monocyclic ring with 5 or 6 ring atoms or a bicyclic ring structure comprising a 6 membered ring fused to a 4, 5, or 6 membered ring; each of the rings being saturated, partially unsaturated, or completely unsaturated; at least one of the rings containing 1 to 4 heteroatoms each independently selected from nitrogen, oxygen and sulphur; and any one of the rings being optionally substituted with one, two or three substituents each independently selected from the group consisting of halo, hydroxy, nitro, cyano, carboxyl, C₁₋₆alkyl, C₁₋₆alkoxy, C₁₋₆alkoxyC₁₋₆alkyl, C₁₋₆alkylcarbonyl, amino, mono- or diC₁₋₆alkylamino, azido, mercapto, polyhaloC₁₋₆alkyl, polyhaloC₁₋₆alkoxy, and C₃₋₇cycloalkyl.

Another embodiment of the present invention relates to libraries of compounds of formula (I) or of any subgroup of compounds of formula (I), wherein one or more of the following conditions apply:
R¹ is hydrogen;
R² is aryl, Het or C₂₋₆alkenyl optionally substituted with C₃₋₇cycloalkyl, aryl or Het;
R³ is C₁₋₆alkyl optionally substituted with C₃₋₇cycloalkyl or aryl, C₁₋₆alkyl optionally substituted with Het, C₃₋₇cycloalkyl optionally substituted with C₁₋₆alkyl, C₂₋₆alkenyl optionally substituted with C₃₋₇cycloalkyl, aryl or Het; aryl; Het;
n is one;
each aryl as a group or part of a group is phenyl or naphthalenyl, each optionally substituted with one, two or three substituents selected from halo, hydroxy, nitro, cyano, carboxyl, C₁₋₆alkyl, C₁₋₆alkoxy, C₁₋₆alkoxyC₁₋₆alkyl, C₁₋₆alkylcarbonyl, amino, mono- or diC₁₋₆alkylamino, azido, mercapto, polyhaloC₁₋₆alkyl, and polyhaloC₁₋₆alkoxy; and
each Het as a group or part of a group is a monocyclic ring with 5 or 6 ring atoms or a bicyclic ring structure comprising a 6 membered ring fused to a 4, 5, or 6 membered ring; each of the rings being saturated, partially unsaturated, or completely unsaturated; at least one of the rings containing 1 to 4 heteroatoms each independently selected from nitrogen, oxygen and sulphur; and any one of the rings being optionally substituted with one, two or three substituents each independently selected from the group consisting of halo, hydroxy, nitro, cyano, carboxyl, C₁₋₆alkyl, C₁₋₆alkoxy, C₁₋₆alkoxyC₁₋₆alkyl, C₁₋₆alkylcarbonyl, amino, mono- or diC₁₋₆alkylamino, azido, mercapto, polyhaloC₁₋₆alkyl, polyhaloC₁₋₆alkoxy, and C₃₋₇cycloalkyl.

The libraries of compounds of the present invention may be prepared according to the procedures described hereinafter, which are meant to be applicable for as well the racemates, stereochemically pure intermediates or end products, or any stereoisomeric mixtures. The racemates or stereochemical mixtures may be separated into stereoisomeric forms at any stage of the synthesis procedures.

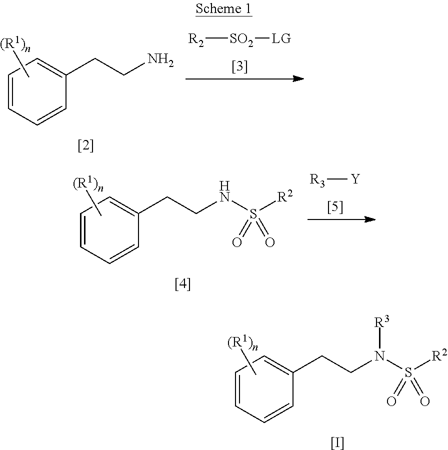

As shown in the above scheme 1, coupling of a compound of formula [2] with compounds of formula R₂—SO₂-LG, where LG means “leaving group”, being said LG group preferably an halogen atom, more preferably bromine or chlorine, yields the corresponding substituted sulfonamides of formula [4]. The reaction solvent is a chlorinated solvent, preferably dichloromethane, 1,2-dichloroethane or chloroform, or a polar aprotic solvent, preferably acetonitrile, tetrahydrofuran, or dimethylformamide, at a temperature preferably between 0° C. and 40° C., more preferably between 10° C. and 25° C.

Under substitution or coupling conditions with compounds of formula R₃—Y, where Y means “leaving group” in substitution reaction and “activating group” in coupling reactions, being said Y preferably is a halogen atom, more preferably bromine or chlorine in substitution reaction, or an activated carboxyl derivative in coupling reactions, compound [4] is converted to the final compounds of formula (I). The reaction solvent is anhydrous or non anhydrous polar aprotic solvent, preferably acetonitrile, tetrahydrofuran, or dimethylformamide, at a temperature preferably between −78° C. and 60° C., more preferably between −78° C. and 25° C.

Both racemic as well as pure enantiomers of (I) can be accessed by this approach depending on the stereochemical integrity of the starting material.

Likewise, an embodiment of the present invention relates to a process for preparing a library of compounds of formula (I) as described herein, said process comprising

a) reacting in a suitable medium compound of formula (II) with a compound of formula (III)

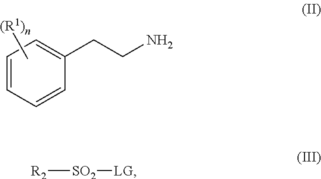

and
b) optionally further reacting in a suitable medium the product of step a) with R₃—Y; wherein
R₁, R₂, R₃, and n have the same definition as provided herein;
LG is a leaving group;
Y is an activating group in coupling reactions or a leaving group in substitution reactions.

The suitable medium of the reaction in step a) is anhydrous or non anhydrous chlorinated solvent, preferably dichloromethane, 1,2-dichloroethane or chloroform, or a anhydrous or non anhydrous polar aprotic solvent, preferably acetonitrile, tetrahydrofuran, or dimethylformamide, at a temperature preferably between 0° C. and 40° C., more preferably between 0° C. and 25° C.

The suitable medium of the reaction in step b) is in the presence of an inorganic or organic base, such as sodium hydride, potassium tert-butoxide or lithium diisopropylamide, at a temperature preferably between −78° C. and 60° C., more preferably between −78° C. and 25° C. The reaction solvent is a polar aprotic solvent, preferably acetonitrile, tetrahydrofuran, dimethylformamide, or dimethylsulfoxide.

The term “leaving group” is preferably a halogen atom, more preferably bromine or chlorine.

The term “activating group” is preferably but not limited to a carboxyl activant in coupling reactions, preferably in the form of an acid chloride, anhydride, or active esters, such as O-acylisoureas or acyloxyphosphonium derivatives.

Compounds of formula (I) making up the libraries may be converted into each other following art-known functional group transformation reactions. For example, amino groups may be N-alkylated, nitro groups reduced to amino groups, a halo atom may be exchanged for another halo.

The libraries of compounds of formula (I) may be converted to the corresponding libraries of N-oxide forms following art-known procedures for converting a trivalent nitrogen into its N-oxide form. Said N-oxidation reaction may generally be carried out by reacting the starting material of formula (I) with an appropriate organic or inorganic peroxide. Appropriate inorganic peroxides comprise, for example, hydrogen peroxide, alkali metal or earth alkaline metal peroxides, e.g. sodium peroxide, potassium peroxide; appropriate organic peroxides may comprise peroxy acids such as, for example, benzenecarbo-peroxoic acid or halo substituted benzenecarboperoxoic acid, e.g. 3-chlorobenzenecarboperoxoic acid, peroxoalkanoic acids, e.g. peroxoacetic acid, alkylhydroperoxides, e.g. tert-butyl hydro-peroxide. Suitable solvents are, for example, water, lower alcohols, e.g. ethanol and the like, hydrocarbons, e.g. toluene, ketones, e.g. 2-butanone, halogenated hydrocarbons, e.g. dichloromethane, and mixtures of such solvents.

Libraries of pure stereochemically isomeric forms of the compounds of formula (I) may be obtained by the application of art-known procedures. Diastereomers may be separated by physical methods such as selective crystallization and chromatographic techniques, e.g., counter-current distribution, liquid chromatography and the like.

The libraries of compounds of formula (I) may be obtained as racemic mixtures of enantiomers which can be separated from one another following art-known resolution procedures. The racemic compounds of formula (I), which are sufficiently basic or acidic may be converted into the corresponding diastereomeric salt forms by reaction with a suitable chiral acid, respectively chiral base. Said diastereomeric salt forms are subsequently separated, for example, by selective or fractional crystallization and the enantiomers are liberated therefrom by alkali or acid. An alternative manner of separating the enantiomeric forms of the compounds of formula (I) involves liquid chromatography, in particular liquid chromatography using a chiral stationary phase. Said pure stereochemically isomeric forms may also be derived from the corresponding pure stereochemically isomeric forms of the appropriate starting materials, provided that the reaction occurs stereospecifically. Preferably if a specific stereoisomer is desired, said compound may be synthesized by stereospecific methods of preparation. These methods may advantageously employ enantiomerically pure starting materials.

From a further point of view, the present invention relates to a pharmaceutical composition including a therapeutically effective amount of a compound of formula (I) as disclosed herein, or a compound of any of the subgroups of compounds of formula (I) as disclosed herein, and a pharmaceutically acceptable vehicle. A therapeutically effective amount in this context is an amount enough for stabilizing, reducing or acting prophylactically against a disease or conditions such as inflammation or coagulation. Furthermore, the present invention relates to a process of preparation of a pharmaceutical composition as disclosed herein, including the admixture of a pharmaceutically acceptable vehicle with a therapeutically effective amount of a compound of formula (I), as disclosed herein, or one of the subgroups of compounds of formula (I) as disclosed herein.

Therefore, the compounds of the present invention or any of the subgroups thereof can be formulated in several pharmaceutical forms with the object of being administered. As suitable composition all compositions typically used for the administration of drugs can be mentioned. In order to prepare a pharmaceutical composition of the present invention, a therapeutically effective amount of the compound in particular, optionally in the form of an addition salt or metallic complex, as an active ingredient, is admixed with the pharmaceutically acceptable vehicle, where the vehicle may be present in several forms depending on the intended route of administration. These pharmaceutical compositions are preferred in the form of a single dosage, particularly, for oral, rectal, percutaneous or parenteral injection administration. For example, in the preparation of compositions for oral single dosage forms, any of the pharmaceutical means can be used such as, for example, water, glycols, oils, alcohols, and the like in the case of liquid oral preparations such as suspensions, syrups, mouthwashes, emulsions and solutions; or solid vehicles, such as starch, sugars, kaolin, lubricants, binding agents, disgregating agents and the like in the case of powders, pills, capsules and tablets. Due to its easy administration, tablets and capsules are the most advantageous forms for single dosage, in which case it is obvious that solid pharmaceutical vehicles are used. For parenteral compositions, the vehicle will often include sterile water, at least mostly, although other ingredients should be included, for example, for improving the solubility. For example, injectable solutions can be prepared where the vehicle comprises saline solutions, glucose solutions or a mixture of saline and glucose solutions. Injectable suspensions can also be prepared in which case suitable vehicle liquid, suspending agents and the like can be used. Also included herein are preparations of solid forms which are intended to convert into liquid preparations just before their use. In the suitable compositions for percutaneous administration, the vehicle optionally comprises an enhancing agent for penetration and/or a suitable wetting agent, optionally combined with suitable additives of any kind in minor quantities, additives which are not significantly deleterious for the skin.

It is particularly advantageous to formulate the above pharmaceutical compositions in the form of a single dosage due to the ease of administration and uniformity in the dosage. The form of single dosage, as previously used, relates to discrete physical units suitable as singles dosages, each dosage containing a predetermined quantity of active ingredient calculated for producing the intended therapeutic effect associated with the required pharmaceutical vehicle. Examples of this type of single dosage form are tablets (including scratched or coated tablets), capsules, pills, suppositories, sachets, wafers, injectable solutions or suspensions and the like, and multiple variations thereof.

Accordingly, the compounds of the present invention or any subgroup thereof can be used as a drug. Said use as a drug or method of treatment comprises administering to an individual an effective amount of the compound of formula (I) for combating conditions associated to different diseases, such as inflammation or coagulation,

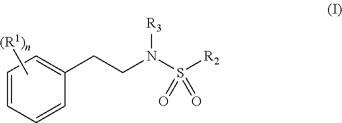

wherein
R¹ is hydrogen, halo, hydroxy, nitro, cyano, carboxyl, C₁₋₆alkyl, C₁₋₆alkoxy, C₁₋₆alkoxyC₁₋₆alkyl, C₁₋₆alkylcarbonyl, amino, mono- or diC₁₋₆alkylamino, azido, mercapto, polyhaloC₁₋₆alkyl, and polyhaloC₁₋₆alkoxy, aryl, Het;
R² is C₁₋₆alkyl, C₁₋₆alkylcarbonyl, C₁₋₆alkyl optionally substituted with aryl, C₁₋₆alkoxyC₁₋₆alkyl, or C₃₋₇cycloalkyl, C₁₋₆alkyl optionally substituted with Het, C₃₋₇cycloalkyl optionally substituted with C₁₋₆alkyl; C₁₋₆alkyl optionally substituted with C₃₋₇cycloalkyl or aryl, C₂₋₆alkenyl optionally substituted with C₃₋₇cycloalkyl, aryl or Het; aryl; Het;
R³ is C₁₋₆alkyl, C₁₋₆alkoxy, C₁₋₆alkoxyC₁₋₆alkyl, C₁₋₆alkylcarbonyl, mono- or diC₁₋₆alkylamino, polyhaloC₁₋₆alkyl, and polyhaloC₁₋₆alkoxy, C₁₋₆alkoxyC₁₋₆alkyl, or C₃₋₇cycloalkyl, C₁₋₆alkyl optionally substituted with C₃₋₇cycloalkyl or aryl, C₁₋₆alkyl optionally substituted with Het, C₃₋₇cycloalkyl optionally substituted with C₁₋₆alkyl, C₂₋₆alkenyl optionally substituted with C₃₋₇cycloalkyl, aryl or Het; aryl, Het, C₁₋₆alkyl optionally substituted with —NR^{4a}R^{4b}, wherein R^{4a} and R^{4b} are, each independently, C₁₋₆alkyl, or R^{4a} and R^{4b} together with the nitrogen to which they are attached form a 5- or 6-membered saturated heterocyclic ring;
n is one, two, three, four or five;
each aryl as a group or part of a group is phenyl or naphthalenyl, each optionally substituted with one, two or three substituents selected from halo, hydroxy, nitro, cyano, carboxyl, C₁₋₆alkyl, C₁₋₆alkoxy, C₁₋₆alkoxyC₁₋₆alkyl, C₁₋₆alkylcarbonyl, amino, mono- or diC₁₋₆alkylamino, azido, mercapto, polyhaloC₁₋₆alkyl, and polyhaloC₁₋₆alkoxy; and
each Het as a group or part of a group is a monocyclic ring with 5 or 6 ring atoms or a bicyclic ring structure comprising a 6 membered ring fused to a 4, 5, or 6 membered ring; each of the rings being saturated, partially unsaturated, or completely unsaturated; at least one of the rings containing 1 to 4 heteroatoms each independently selected from nitrogen, oxygen and sulphur; and any one of the rings being optionally substituted with one, two or three substituents each independently selected from the group consisting of halo, hydroxy, nitro, cyano, carboxyl, C₁₋₆alkyl, C₁₋₆alkoxy, C₁₋₆alkoxyC₁₋₆alkyl, C₁₋₆alkylcarbonyl, amino, mono- or diC₁₋₆alkylamino, azido, mercapto, polyhaloC₁₋₆alkyl, polyhaloC₁₋₆alkoxy, and C₃₋₇cycloalkyl.

Similarly, the present invention relates to compounds of formula (I) included in the previous paragraph for use in the treatment of diseases or conditions such as inflammation or coagulation.

In addition, the present invention relates to a method of treatment a disease or conditions such as inflammation or coagulation warm-blooded animals, said method including administering an effective amount of compound of formula (I) as indicated in the previous paragraphs, or of a compound from any of the compounds of formula (I).

The term “therapeutically effective amount” as used herein is referred to the amount of a compound or component or active pharmaceutical agent for obtaining a biological or medicinal response in the tissue, system, animal or human being investigated, in the view of the present invention, by an investigator, veterinary, physician or other clinical attendant, including the relief of the symptoms of the disease being treated.

### EXAMPLES

The following examples are intended to illustrate the present invention and not to limit it thereto.

### Example 1

### Preparation of N-phenethylbenzenesulfonamide

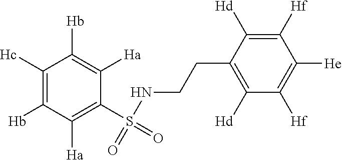

To a stirred solution of 2-phenylethylamine (2 g, 16.5 mmol) in 105 ml DMF at room temperature was added Et₃N (2.75 ml, 19.8 mmol). This mixture was stirred for 5 min, and then benzenesulfonyl chloride (2.3 ml, 18.1 mmol) was added at this temperature. The reaction was stirred for 2.5 h, and then, the mixture was evaporated to dryness and the crude was chromatographically purified over SiO₂ using Hexane/AcOEt 60/40 as the eluant, yielding 3.12 g (72%) of the desired product.

¹H-NMR (400 MHz, DMSO-d₆): 7.79 (d, 2H, J=7.6 Hz, Hₐ), 7.7 (sa, 1H, NH), 7.60 (m, 3H, 2H_{b}+H_{c}), 7.26 (t, 2H, J=7.6 Hz, H_{d}), 7.19 (m, 1H, Hₑ), 7.15 (m, 2H, H_{f}), 2.9 (t, 2H, J=7.3 Hz, NHCH₂CH₂), 2.6 (t, 2H, J=7.4 Hz, NHCH₂CH₂) ppm.

CAS nr: [77198-99-3]

### Example 2

### Preparation of N-phenethylnaphthalene-1-sulfonamide

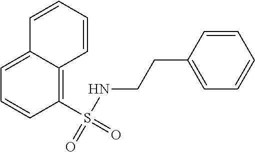

To a stirred solution of 2-phenylethylamine (3.5 mmol, 1 eq) in 40 ml CH₂Cl₂ were consecutively added, Et₃N (0.58 ml, 4.18 mmol, 1.2 eq) and the corresponding sulfonyl chloride (2-naphthalenesulfonyl chloride, 0.87 g, 3.84 mmol, 1.1 eq). The reaction was performed at room temperature during 4 h, until the total consumption of the starting material. Once the solvent was evaporated, the crude mixture was chromatographically purified over Al₂O₃ using Hexane/AcOEt (70:30) as the eluant. The final yield of the product obtained was 91%, and the purity≧99% (expressed in % HPLC area).

¹H-NMR (400 MHz, CDCl₃): 8.39-7.61 (m, 7H, H_{Ar naph}), 7.25-7.04 (m, 5H, H_{Ar Phe}), 4.43 (sa, 1H, NH), 3.27 (c, 2H, J=6.3 Hz, NHCH₂CH₂), 2.77 (t, 2H, J=6.6 Hz, NHCH₂CH₂) ppm.

CAS nr: [126402-52-6]

### Example 3

### Preparation of 4-Chloro-N-phenethylbenzenesulfonamide

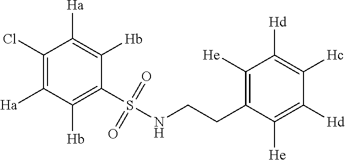

Following a procedure analogous to that described in Example 2, using 4-chlorobenzenesulfonyl chloride as the starting material, the title compound was obtained in 91% yield (Purity≧99%).

¹H-NMR (400 MHz, CDCl₃): 7.74 (d, 2H, J=4.7 Hz, H_{b}), 7.47 (d, 2H, J=4.7 Hz, Hₐ), 7.28 (m, 3H, 2Hₑ+H_{c}), 7.10 (m, 2H, H_{d}), 4.46 (t, 1H, J=6 Hz, NH), 3.26 (c, 2H, J=6.7 Hz, NHCH₂CH₂), 2.80 (t, 2H, J=6.8 Hz, NHCH₂CH₂) ppm.

CAS nr: [133276-82-1]

### Example 4

### Preparation of N-phenethylquinoline-8-sulfonamide

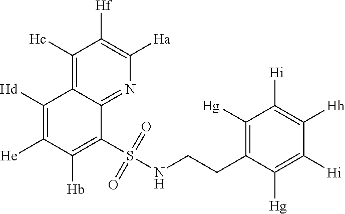

Following a procedure analogous to that described in Example 2, using 8-quinolinesulfonyl chloride as the starting material, the title compound was obtained in 93% yield (Purity≧99%).

¹H-NMR (400 MHz, CDCl₃): 8.64 (dd, 1H, ³J_{a-f}=4.3 Hz, ⁴J_{a-c}=1.7 Hz, Ha), 8.42 (dd, 1H, ³J_{b-e}=7.3 Hz, ⁴J_{b-d}=1.2 Hz, Hb), 8.23 (dd, 1H, ³J_{c-f}=8.3 Hz, ⁴J_{c-a}=1.7 Hz, Hc), 8.03 (dd, 1H, ³J_{d-e}=8.2 Hz, ⁴J_{d-b}=1.2 Hz, Hd), 7.64 (dd, 1H, ³J_{e-d}=8 Hz, ³J_{e-b}=7.6 Hz, He), 7.46 (dd, 1H, ³J_{f-c}=8.3 Hz, ³J_{f-a}=4.3 Hz, Hf), 7.14 (m, 3H, Hg+Hh), 6.95 (m, 2H, Hi), 6.35 (t, 1H, J=5.8 Hz, NH), 3.15 (c, 2H, J=6.6 Hz, NHCH₂), 2.76 (t, 2H, J=6.5 Hz, NHCH₂CH₂) ppm.

CAS nr: [289500-01-2]

### Example 5

### Preparation of 5-(Dimethylamino)-N-phenethylnaphthalene-1-sulfonamide

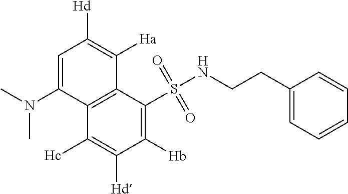

Following a procedure analogous to that described in Example 2, using dansyl chloride as the starting material, the title compound was obtained in 95% yield (Purity≧99%).

¹H-NMR (400 MHz, CDCl₃): 8.55 (d, 1H, J=8.6 Hz, Ha), 8.24 (dd, 1H, ³J_{b-d}=7.3 Hz, ⁴J_{b-c}=1.3 Hz, Hb), 8.17 (d, 1H, J=8.6 Hz, Hc), 7.50 (m, 2H, Hd+Hd′), 7.16 (m, 4H, H_{Ar}), 6.93 (m, 2H, H_{Ar}), 4.62 (t, 1H, J=6.2 Hz, NH), 3.17 (c, 2H, J=6.5 Hz, NHCH₂), 2.89 (s, 6H, N(CH₃)₂), 2.65 (t, 2H, J=6.9 Hz, NHCH₂CH₂) ppm.

CAS nr: [5282-81-5]

### Example 6

### Preparation of (E)-N-phenethyl-2-phenylethenesulfonamide

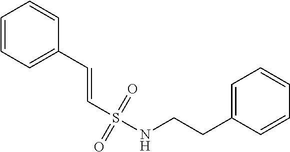

Following a procedure analogous to that described in Example 2, using trans-omega-styrenesulfonyl chloride as the starting material, the title compound was obtained in 75% yield (Purity≧94%).

¹H-NMR (400 MHz, CDCl₃): 7.43 (m, 6H, H_{Ar}), 7.29 (m, 2H, H_{Ar}), 7.24 (d, 1H, ³J_{a-b}=15.1 Hz, Ha), 7.18 (m, 2H, H_{Ar}), 6.60 (d, 1H, ³J_{b-a}=15.4 Hz, Hb), 4.37 (t, 1H, J=6.1 Hz, NH), 3.35 (c, 2H, J=6.7 Hz, NHCH₂), 2.88 (t, 2H, J=6.8 Hz, NHCH₂CH₂) ppm.

CAS nr: [464902-17-8]

### Example 7

### Preparation of 5-chloro-N-phenethylthiophene-2-sulfonamide

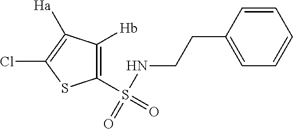

Following a procedure analogous to that described in Example 2, using 5-chlorothiophene-2-sulfonyl chloride as the starting material, the title compound was obtained in 98% yield (Purity≧97%).

¹H-NMR (400 MHz, CDCl₃): 7.33 (d, 1H, ³J_{a-b}=4 Hz, Ha), 7.30-7.11 (m, 5H, H_{Ar}), 6.89 (d, 1H, ³J_{b-a}=4 Hz, Hb), 4.59 (t, 1H, J=5.8 Hz, NH), 3.31 (c, 2H, J=6.7 Hz, NHCH₂), 2.82 (t, 2H, J=6.9 Hz, NHCH₂CH₂) ppm.

CAS nr: [900407-92-3]

### Example 8

### Preparation of N-(2-(1H-indol-3-yl)ethyl)-N-phenethylnaphthalene-1-sulfonamide

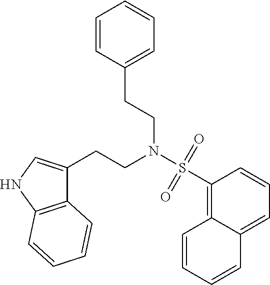

Under inert atmosphere, to a stirred solution of NaH (0.016 g, 0.36 mmol) in 0.20 ml anhydrous DMF at 0° C., was added a solution of compound of Example 2 (0.108 g, 0.35 mmol) in 0.60 ml anhydrous DMF. The temperature was maintained during 1.5 h. After this time, a solution of 3-(2-Bromoethyl)indole (0.088 g, 0.39 mmol) in 0.6 anhydrous DMF was added dropwise to the reaction mixture of step at 0° C., and was stirred during 2.5 h. Once the solvent was evaporated, the crude mixture was chromatographically purified over SiO₂ using Hexane/AcOEt (70:30) as the eluant, furnishing 133 mg of the desired product in 84% yield (purity≧80%, expressed in % HPLC area).

FT-IR (KBr): 3408 cm⁻¹

¹H-RMN (400 MHz, CD₃OD): 8.38 (m, 1H, H_{Ar-Naf}), 7.98 (m, 3H, H_{Ar-Naf}), 7.76 (m, 1H, H_{Ar-Naf}), 7.64 (m, 2H, H_{Ar-Naf}), 7.41 (m, 1H, H_{Ar-Ph}), 7.28 (m, 1H, H_{Ar-Ind}), 7.20-6.92 (m, 8H, 4H_{Ar-Ph}+4H_{Ar-Ind}), 3.50 (t, 2H, J=7.4 Hz, NCH₂CH₂Ind), 3.12 (t, 2H, J=7.4 Hz, NCH₂CH₂Ph), 2.93 (t, 2H, J=7.4 Hz, NCH₂CH₂Ind), 2.70 (t, 2H, J=7.7 Hz, NCH₂CH₂Ph) ppm.

### Example 9

### Preparation of (E)-N-((1-methylpiperidin-3-yl)methyl)-N-phenethyl-2-phenylethenesulfonamide

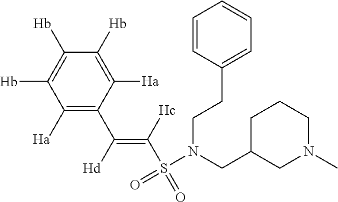

Step 1: Under inert atmosphere, to a stirred solution of NaH (20 mg, 0.45 mmol) in 0.20 ml anhydrous DMF at 0° C., was added a solution of compound of Example 6 (0.101 g, 0.20 mmol) in 0.60 ml anhydrous DMF. The temperature was maintained during 1.5 h.

Step 2: Simultaneously, in another reaction vessel, NaOH 0.1M was added dropwise up to pH=12.5. The aqueous phase was extracted with CHCl₃/IPA 3:1 (3×2 ml). Once the solvent was completely removed, the obtained product was 10 mg (0.067 mmol) of the free base reactant. The solid was dissolved in 0.6 ml of anhydrous DMF.

Step 3: After 1.5 h, the solution of step 2 was slowly added to the solution of step 1, stirring at 0° C. during 2.5 h. The final product crystallized in the solvent when the crude mixture was maintained overnight at −18° C. The obtained solid was in vacuum filtered and washed with acetone at 0° C., furnishing 16 mg (60% yield) of the desired product.

¹H-NMR (400 MHz, CDCl₃): 7.51 (m, 2H, Ha), 7.38 (m, 3H, Hb), 7.30 (d, 1H, J=15.6 Hz, Hc), 7.18 (m, 5H, NHCH₂CH₂Ph), 6.8 (d, 1H, J=15.6 Hz, Hd), 3.18 (m, 2H, NHCH₂), 2.81 (t, 2H, J=7.3 Hz, NHCH₂CH₂) ppm.

### Example 10

### Preparation of 5-chloro-N-((2-(diaminomethyleneamino)thiazol-4-yl)methyl)-N-phenethylthiophene-2-sulfonamide

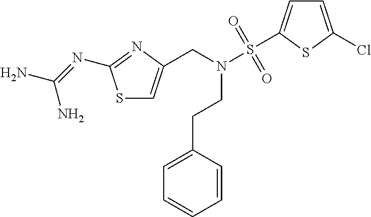

Step 1: Under inert atmosphere, to a stirred solution of NaH (9 mg, 0.19 mmol) in 0.2 ml anhydrous DMF at 0° C., was added a solution of compound of Example 7 (0.053 g, 0.17 mmol) in 0.50 ml anhydrous DMF. The temperature was maintained during 1.5 h.

Step 2: After the first 1 h of step 1, in another reaction vessel, buthyllithium (2.2 eq) was added dropwise to a solution of 1-(4-Bromomethyl-2-thiazoyl)guanidine, hydrobromide salt (45 mg, 0.20 mmol) in 0.5 ml anhydrous DMF at −70° C. The reaction was stirred during 15 minutes.

Step 3: After 1.5 h of step 1 and 15 minutes of step 2, the solution of step 2 was slowly added to the solution of step 1, stirring at 0° C. during 2.5 h.

The crude mixture was diluted in a proportion ⅕H₂O/DMF and chromatographically purified using preparative HPLC in reverse phase conditions, using MeOH/H₂O 65/35 as the mobile phase. The eluant was completely evaporated, furnishing 48 mg (60% yield) of the desired product.

¹H-NMR (400 MHz, CDCl₃): 7.24 (m, 4H_{Ar}), 7.07 (d, 2H, 1H_{Ar}+1Hₜₕᵢₒₚₕₑₙ), 6.89 (d, 1H, J=4 Hz, Hₜₕᵢₒₚₕₑₙ), 6.65 (sa, 1H, H_{thiazole}), 4.30 (s, 2H, NCH_{2 thiazole}), 3.41 (t, 2H, J=7.5 Hz, NCH₂CH₂Ph), 2.80 (t, 2H, J=7.5 Hz, NCH₂CH₂Ph) ppm.

MS: Positive mode [M+H⁺]=459.3

### Example 11

### Preparation of (E)-N-((2-guanidinothiazol-4-yl)methyl)-N-phenethyl-2-phenylethenesulfonamide

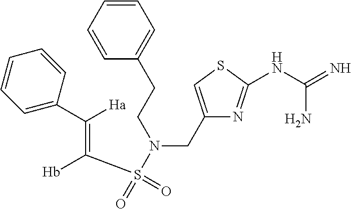

Step 1: Under inert atmosphere, to a stirred solution of NaH (10 mg, 0.24 mmol) in 0.2 ml anhydrous DMF at 0° C., was added a solution of compound of Example 6 (0.053 g, 0.19 mmol) in 0.50 ml anhydrous DMF. The temperature was maintained during 1.5 h.

Step 2: After the first 1 h of step 1, in another reaction vessel, buthyllithium (2.3 eq) was added dropwise to a solution of 1-(4-Bromomethyl-2-thiazoyl)guanidine, hydrobromide salt (50 mg, 0.22 mmol) in 0.5 ml anhydrous DMF at −10° C. The reaction was stirred during 15 minutes.

Step 3: After 1.5 h of step 1 and 15 minutes of step 2, the solution of step 2 was slowly added to the solution of step 1, stirring at 0° C. during 2.5 h.

The crude mixture was diluted in a proportion ⅕H₂O/DMF and chromatographically purified using preparative HPLC in reverse phase conditions, using MeOH/H₂O 65/35 as the mobile phase. The eluant was completely evaporated, furnishing 30 mg (42% yield) of the desired product.

¹H-NMR (400 MHz, CDCl₃): 7.60 (˜d, 2H, H_{Ar}), 7.41 (m, 2H, H_{Ar}), 7.37 (d, 1H, ³J_{a-b}=15.7 Hz, Ha), 7.30-7.12 (m, 6H, H_{Ar}), 6.94 (d, 1H, ³J_{b-a}=15.5 Hz, Hb), 6.65 (sa, 1H, H_{thiazol}), 4.33 (s, 2H, NCH₂ thiazol), 3.42 (t, 2H, J=7.6 Hz, NCH₂CH₂Ph), 2.92 (t, 2H, J=7.5 Hz, NCH₂CH₂Ph) ppm.

### Comparative Example 12

### Preparation of Parent Compound N-Phenethyl-cyclohexanesulfonamide

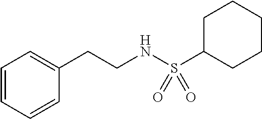

To a stirred solution of 2-phenylethylamine (0.60 g, 4.97 mmol) in 45 ml CH₂Cl₂ at room temperature was added Et₃N (0.83 ml, 5.97 mmol). The mixture was stirred for 5 min, and then cyclohexanesulfonyl chloride (1.0 g, 5.47 mmol) was added at this temperature.

The reaction was stirred for 7 h, and then, the mixture was evaporated to dryness. The crude was chromatographically purified over SiO₂ using Hexane/AcOEt as the eluant, yielding 1.02 g (76%) of the desired product.

¹H-NMR (400 MHz, CDCl₃): 7.4-7.2 (m, 5H, H_{Ar}), 4.3 (sa, 1H, NH), 3.40 (td, 2H, CH₂CH₂NH), 2.88 (t, 2H, J=6.8 Hz, CH₂CH₂NH), 2.77 (m, 1H, —CH—), 2.07 (m, 2H, -2×H_{cyclohexyl}), 1.85 (m, 2H, 2×H_{cyclohexyl}), 1.69 (m, 1H, 1×H_{cyclohexyl}), 1.42 (m, 2H, 2×H_{cyclohexyl}), 1.20 (m, 3H, 3×H_{cyclohexyl}) ppm.

MS: Negative mode [M−H]⁻=265.9

### Example 13

### Preparation of N-(Cyclohexylsulfonyl)-N-Phenethylpivalamide

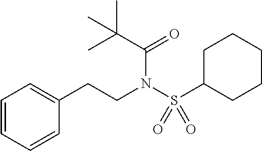

Step 1: Following a procedure analogous to that described in Comparative example 12, using cylohexanesulfonyl chloride as the starting material, the parent compound N-phenethylcyclohexanesulfonamide was obtained in 70% yield.

Step 2: Under inert atmosphere, to a stirred solution of NaH (13 mg, 0.29 mmol) in 0.5 ml anhydrous DMF at 0° C., was added a solution of compound N-phenethylcyclohexanesulfonamide (0.053 g, 0.20 mmol) in 0.50 ml anhydrous DMF.

The temperature was maintained during 1.5 h.

Then, pivaloyl chloride (1.2 eq) was added dropwise at 0° C. and the reaction was warmed to room temperature while stirring during 2.5 h.

Once the solvent was evaporated to dryness, the crude mixture was chromatographically purified over SiO₂ using mixtures of Hexane and AcOEt as the eluant, furnishing the expected product in 25% yield.

¹H-NMR (400 MHz, CDCl₃): 7.4-7.2 (m, 5H, H_{Ar}), 3.9 (m, 2H, CH₂CH₂NH), 3.6 (m, 1H, —CH_{cyclohexyl}), 3.1 (t, 2H, J=8.4 Hz, CH₂CH₂NH), 2.2 (m, 2H, 2×H_{cyclohexyl}), 2.1 (m, 3H, 3×H_{cyclohexyl}), 1.9-1.4 (m, 5H, 5×H_{cyclohexyl}), 1.39 (s, 9H, 3×CH₃, ^{t}Bu) ppm.

MS: Positive mode [M+Na]⁺=374.2

### Example 14

### Preparation of (E)-Methyl phenethyl(Styrylsulfonyl)carbamate

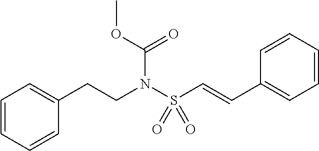

Step 1: Following a procedure analogous to that described in Comparative example 12, using trans-omega-styrenesulfonyl chloride as the starting material, the parent compound (E)-N-phenethyl-2-phenylethenesulfonamide was obtained in 75% yield.

Step 2: Under inert atmosphere, to a stirred solution of NaH (23 mg, 0.52 mmol) in 0.5 ml anhydrous DMF at 0° C., was added a solution of compound (E)-N-phenethyl-2-phenylethenesulfonamide (0.10 g, 0.35 mmol) in 1.0 ml anhydrous DMF. The temperature was maintained during 1.5 h.

Then, methyl chloroformate (1.2 eq) was added dropwise at 0° C. and the reaction was warmed to room temperature while stirring during 2.5 h.

Once the solvent was evaporated to dryness, the crude mixture was chromatographically purified over SiO₂ using Hexane/AcOEt (80:20) as the eluant, furnishing 36.2 mg of the expected product in 30% yield.

¹H-NMR (400 MHz, CDCl₃): 7.5 (d, 1H, J=15.5 Hz, —CH═CH-Ph), 7.5-7.2 (m, 10H, 10×H_{Ar}), 6.8 (d, 1H, J=15.4 Hz, —CH═CH-Ph), 4.0 (t, 2H, J=6.4 Hz, CH₂CH₂NH), 3.8 (s, 3H, COOCH₃), 3.0 (t, 2H, J=6.3 Hz, CH₂CH₂NH), 2.8 (m, 1H, CH), 1.3 (m, 2H, 2×H_{cyclopropyl}), 1.1 (m, 2H, 2×H_{cyclopropyl}) ppm.

MS: Positive mode [M+Na]⁺=368.1

### Example 15

### Preparation of N-(Methylsulfonyl)-N-Phenethylacetamide

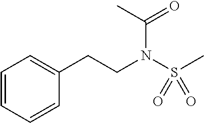

Step 1: Following a procedure analogous to that described in comparative example 12, using methanesulfonyl chloride as the starting material, the parent compound was obtained in 96% yield.

Step 2: Following a procedure analogous to that described in Examples 13 and 14, using acetyl chloride as the starting material, the title compound was obtained in 35% yield.

¹H-NMR (400 MHz, CDCl₃): 7.4-7.2 (m, 5H, H_{Ar}), 4.0 (t, 2H, J=7.6 Hz, CH₂CH₂NH), 3.1 (s, 3H, —SO₂CH₃), 3.00 (t, 2H, J=7.5 Hz, CH₂CH₂NH), 2.3 (s, 3H, —CO—CH₃) ppm.

MS: Positive mode [M+Na]⁺=263.9

### Example 16

### Preparation of N-phenethyl-N-(propylsulfonyl)acetamide

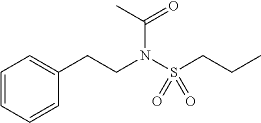

Step 1: Following a procedure analogous to that described in comparative example 12, using 1-propanesulfonyl chloride as the starting material, the parent compound was obtained in 80% yield.

Step 2: Following a procedure analogous to that described in Examples 13 and 14, using acetyl chloride as the starting material, the title compound was obtained in 30% yield.

¹H-NMR (400 MHz, CDCl₃): 7.3-7.1 (m, 5H, H_{Ar}), 3.8 (t, 2H, J=6.6 Hz, CH₂CH₂NH), 3.2 (t, 2H, J=7.0 Hz, SO₂—CH₂CH₂CH₃), 2.9 (t, 2H, J=6.4 Hz, CH₂CH₂NH), 2.4 (s, 3H, CO—CH₃), 1.7 (m, 2H, SO₂—CH₂CH₂CH₃), 1.0 (s, 3H, SO₂—CH₂CH₂CH₃) ppm.

MS: Positive mode [M+Na]⁺=306.0

### Example 17

### Preparation of N-(cyclopropylsulfonyl)-2-methoxy-N-phenethylacetamide

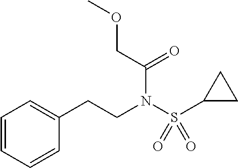

Step 1: Following a procedure analogous to that described in comparative example 12, using cyclopropanesulfonyl chloride as the starting material, the parent compound was obtained in 94% yield.

Step 2: Following a procedure analogous to that described in Example 13 and 14, using acetyl chloride as the starting material, the title compound was obtained in 58% yield.

¹H-NMR (400 MHz, CDCl₃): 7.3-7.2 (m, 5H, H_{Ar}), 4.2 (s, 2H, CO—CH₂—O—), 3.8 (t, 2H, J=6.7 Hz, CH₂CH₂NH), 3.4 (s, 3H, —O—CH₃), 3.0 (t, 2H, J=6.7 Hz, CH₂CH₂NH), 2.8 (m, 1H, CH), 1.3 (m, 2H, 2×H_{cyclopropyl}), 1.1 (m, 2H, 2×H_{cyclopropyl}) ppm.

MS: Positive mode [M+Na]⁺=320.0

What is claimed is:

1. A library of chemical compounds wherein each member of said library is a compound of formula (I) 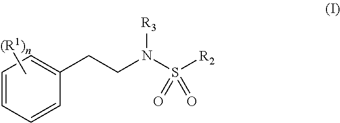
and the salts and stereoisomers thereof, wherein
R¹ is hydrogen, halo, hydroxy, nitro, cyano, carboxyl, C₁₋₆alkyl, C₁₋₆alkoxy, C₁₋₆alkoxyC₁₋₆alkyl, C₁₋₆alkylcarbonyl, amino, mono- or diC₁₋₆alkylamino, azido, mercapto, polyhaloC₁₋₆alkyl, and polyhaloC₁₋₆alkoxy, aryl, Het;
R² is C₁₋₆alkyl, C₁₋₆alkylcarbonyl, C₁₋₆alkoxyC₁₋₆alkyl, or C₃₋₇cycloalkyl, C₃₋₇cycloalkyl optionally substituted with C₁₋₆alkyl, C₁₋₆alkyl optionally substituted with C₃₋₇cycloalkyl, aryl or Het, C₂₋₆alkenyl optionally substituted with C₃₋₇cycloalkyl or aryl; aryl; Het;
R³ is C₁₋₆alkylcarbonyl, polyhaloC₁₋₆alkyl, polyhaloC₁₋₆alkoxy, C₁₋₆alkoxyC₁₋₆alkyl, C₁₋₆alkyl substituted with C₃₋₇cycloalkyl, C₂₋₆alkenyl substituted with C₃₋₇cycloalkyl, aryl or Het;
n is one, two, three, four or five;
each aryl as a group or part of a group is phenyl or naphthalenyl, each optionally substituted with one, two or three substituents selected from halo, hydroxy, nitro, cyano, carboxyl, C₁₋₆alkyl, C₁₋₆alkoxy, C₁₋₆alkoxyC₁₋₆alkyl, C₁₋₆alkylcarbonyl, amino, mono- or diC₁₋₆alkylamino, azido, mercapto, polyhaloC₁₋₆alkyl, and polyhaloC₁₋₆alkoxy; and
each Het as a group or part of a group is a monocyclic ring with 5 or 6 ring atoms or a bicyclic ring structure comprising a 6 membered ring fused to a 4, 5, or 6 membered ring; each of the rings being saturated, partially unsaturated, or completely unsaturated; at least one of the rings containing 1 to 4 heteroatoms each independently selected from nitrogen, oxygen and sulphur; and any one of the rings being optionally substituted with one, two or three substituents each independently selected from the group consisting of halo, hydroxy, nitro, cyano, carboxyl, C₁₋₆alkyl, C₁₋₆alkoxy, C₁₋₆alkoxyC₁₋₆alkyl, C₁₋₆alkylcarbonyl, amino, mono- or diC₁₋₆alkylamino, azido, mercapto, polyhaloC₁₋₆alkyl, polyhaloC₁₋₆alkoxy, and C₃₋₇cycloalkyl.

2. The library of compounds according to claim 1, wherein
R¹ is hydrogen, aryl, Het;
R² is C₃₋₇cycloalkyl optionally substituted with C₁₋₆alkyl, C₁₋₆alkyl optionally substituted with C₃₋₇cycloalkyl or aryl or Het, C₂₋₆alkenyl optionally substituted with C₃₋₇cycloalkyl, aryl; aryl; Het;
R³ is C₁₋₆-alkylcarbonyl, polyhaloC₁₋₆alkyl, polyhaloC₁₋₆alkoxy, C₁₋₆alkoxyC₁₋₆alkyl, C₁₋₆alkyl substituted with C₃₋₇cycloalkyl, C₂₋₆alkenyl substituted with C₃₋₇cycloalkyl, aryl or Het;
n is one or two;
each aryl as a group or part of a group is phenyl or naphthalenyl, each optionally substituted with one, two or three substituents selected from halo, hydroxy, nitro, cyano, carboxyl, C₁₋₆-alkyl, C₁₋₆alkoxy, C₁₋₆alkoxyC₁₋₆alkyl, C₁₋₆alkylcarbonyl, amino, mono- or diC₁₋₆alkylamino, azido, mercapto, polyhaloC₁₋₆alkyl, and polyhaloC₁₋₆alkoxy; and
each Het as a group or part of a group is a monocyclic ring with 5 or 6 ring atoms or a bicyclic ring structure comprising a 6 membered ring fused to a 4, 5, or 6 membered ring; each of the rings being saturated, partially unsaturated, or completely unsaturated; at least one of the rings containing 1 to 4 heteroatoms each independently selected from nitrogen, oxygen and sulphur; and any one of the rings being optionally substituted with one, two or three substituents each independently selected from the group consisting of halo, hydroxy, nitro, cyano, carboxyl, C₁₋₆alkyl, C₁₋₆alkoxy, C₁₋₆alkoxyC₁₋₆alkyl, C₁₋₆alkylcarbonyl, amino, mono- or diC₁₋₆alkylamino, azido, mercapto, polyhaloC₁₋₆alkyl, polyhaloC₁₋₆alkoxy, and C₃₋₇cycloalkyl.

3. The library of compounds according to claim 1, wherein
R¹ is hydrogen;
R² is aryl or Het;
R³ is C₁₋₆alkylcarbonyl, polyhaloC₁₋₆alkyl, polyhaloC₁₋₆alkoxy, C₁₋₆alkoxyC₁₋₆alkyl, C₁₋₆alkyl substituted with C₃₋₇cycloalkyl, C₂₋₆alkenyl optionally substituted with C₃₋₇cycloalkyl, aryl or Het;
n is one;
each aryl as a group or part of a group is phenyl or naphthalenyl, each optionally substituted with one, two or three substituents selected from halo, hydroxy, nitro, cyano, carboxyl, C₁₋₆alkyl, C₁₋₆alkoxy, C₁₋₆alkoxyC₁₋₆alkyl, C₁₋₆alkylcarbonyl, amino, mono- or diC₁₋₆alkylamino, azido, mercapto, polyhaloC₁₋₆alkyl, and polyhaloC₁₋₆alkoxy; and
each Het as a group or part of a group is a monocyclic ring with 5 or 6 ring atoms or a bicyclic ring structure comprising a 6 membered ring fused to a 4, 5, or 6 membered ring; each of the rings being saturated, partially unsaturated, or completely unsaturated; at least one of the rings containing 1 to 4 heteroatoms each independently selected from nitrogen, oxygen and sulphur; and any one of the rings being optionally substituted with one, two or three substituents each independently selected from the group consisting of halo, hydroxy, nitro, cyano, carboxyl, C₁₋₆alkyl, C₁₋₆alkoxy, C₁₋₆alkoxyC₁₋₆alkyl, C₁₋₆alkylcarbonyl, amino, mono- or diC₁₋₆alkylamino, azido, mercapto, polyhaloC₁₋₆alkyl, polyhaloC₁₋₆alkoxy, and C₃₋₇cycloalkyl.

4. A pharmaceutical composition comprising a vehicle and as an active ingredient a therapeutic amount of a compound as defined in any of claims 1 to 3.

5. A method of treatment comprising administering to an individual an effective amount of a compound of formula (I) according to any of claims 1-3, or salts or stereoisomers thereof, for combating conditions associated with a disease or condition.

6. The method according to claim 5 in the treatment of diseases or conditions related to inflammation or coagulation.

7. The method according to claim 5 for treatment of a disease or condition in a warm-blooded animal.
